(19) United States
(12) Patent Application Publication
(10) Pub. No.: US 2026/0133169 A1
(43) Pub. Date: May 14, 2026

(54) STATION AND METHOD FOR MEASURING AIRBORNE MOLECULAR CONTAMINATION

(71) Applicant: PFEIFFER VACUUM, Annecy (FR)

(72) Inventors: Julien BOUNOUAR, Annecy Cedex (FR); Stéphane MARCHAL, Annecy Cedex (FR); Marie DE VITA, Annecy Cedex (FR); Arnaud ALEVEQUE, Annecy Cedex (FR)

(73) Assignee: PFEIFFER VACUUM, Annecy (FR)

(21) Appl. No.: 19/119,449

(22) PCT Filed: Sep. 6, 2023
(86) PCT No.: PCT/EP2023/074478

§ 371 Date: Apr. 9, 2025

(30) Foreign Application Priority Data
Oct. 24, 2022 (FR) FR2211025

(51) Int. Cl. G01N 33/00

(52) U.S. Cl. CPC G01N 33/0022; G01N 33/0006; G01N 33/0072; G01N 33/0073

(57) ABSTRACT

A station for measuring airborne molecular contamination includes at least one gas analyzer to measure a concentration of at least one contaminant, a plurality of air inlets to be in fluidic communication with the gas analyzer and to be connected to a respective plurality of sampling lines, a plurality of first controllable isolation valves interposed between the plurality of air inlets and the gas analyzer, and a control unit to control the opening or the closure of the first controllable isolation valves in order to place the gas analyzer in fluidic communication with at least one sampling line. The control unit also commands a verification of the accuracy of the analyzer when the concentration of a contaminant greater than a predetermined threshold is detected by the analyzer.

The present invention relates to a station for measuring airborne molecular contamination, intended in particular for monitoring the molecular contamination in the atmosphere of clean rooms, such as the clean rooms of semiconductor fabrication factories. The present invention also relates to a method for detecting airborne molecular contamination by means of such a measurement station.

In the semiconductor fabrication industry, substrates, such as semiconductor wafers or photomasks, have to be protected from airborne molecular contamination (or AMC) in order to prevent the latter from damaging the chips or electronic circuits of the substrates. To this end, the wafers are placed in transport and storage boxes, allowing the wafers to be transported from one item of equipment to the other or to be stored between two fabrication steps. Furthermore, the transport boxes and the items of equipment are arranged inside clean rooms in which the contamination level is controlled and the temperature, humidity and pressure are maintained at precise and stable levels.

In clean rooms, airborne gaseous species can have different sources and different natures, for example acids, bases, alcohols, condensable elements, doping elements, etc. These molecules may originate from the air inside the semiconductor fabrication factory or may result from degassing of the wafers subsequent to a fabrication operation.

Gas analysers present in the clean rooms allow the concentration of airborne gaseous species, notably that of humidity, to be evaluated in real time. The measured concentrations are sometimes very low, of the order of ppm or down to the sub-ppb level.

Since these gas analysers measure the surrounding gaseous atmosphere, it is therefore necessary to provide a gas analyser in each zone to be tested of the clean room.

There is a need to increase the number of gaseous species measured and the number of test zones in order to reduce the risks of contamination of the substrates. However, multiplying the number of analysers per zone and multiplying the number of these zones to be tested rapidly makes this solution very costly.

To reduce the costs, a measurement unit that combines different analysers has been proposed. The unit is provided with a number of inlet ports which each address a particular test zone of the clean room via sampling lines.

However, as the sampling lines have different lengths and the concentrations of contaminants can be very low, erroneous measurements may occur and in particular false positives may be detected. These false positives can lead to the stoppage of the fabrication of semiconductors and the intervention of an operator in the zone in which the contamination has been detected. This then entails significant costs.

It is therefore necessary to find a solution which makes it possible to reduce or avoid the false positives and which makes it possible to improve the reliability of the detection of contamination.

One of the aims of the present invention is to propose a measurement station and method which at least partially resolve one of the above-mentioned drawbacks.

To that end, a subject of the invention is a station for measuring airborne molecular contamination, comprising:
at least one gas analyser configured to measure a concentration of at least one contaminant,
a plurality of air inlets configured to be in fluidic communication with the gas analyser and configured to be connected to one or more sampling lines,
a plurality of first controllable isolation valves interposed between the plurality of air inlets and the gas analyser,
a control unit configured to control the opening and the closure of the first controllable isolation valves in order to place the gas analyser in fluidic communication with at least one sampling line,
wherein the control unit is also configured to command a verification of the accuracy of the analyser when the concentration of a contaminant greater than a predetermined threshold is detected by said analyser.

Carrying out a verification of the accuracy of the analyser when the concentration of a contaminant greater than a predetermined threshold is measured makes it possible to limit the number of instances of incorrect contamination detection and thus to simplify the management of the contamination of a clean room.

According to another aspect of the present invention, the measurement station also comprises a reservoir comprising a reference fluid having a predetermined concentration of one or more contaminants to be monitored and an additional valve disposed between the reservoir and the gas analyser, the control unit being configured to, during the verification of the accuracy of the analyser, control the opening of the additional valve and to command a measurement of the concentration of one or more contaminants in the reservoir.

The verification of the accuracy may be effected by injection of a clean fluid or gas (devoid of contaminant) or of a reference fluid or gas having a controlled (known) concentration of one or more contaminants (the reference gas may contain one or more compounds). The reference fluid or gas may come from a permeation tube, which may or may not be heated. The measurement station may also comprise means for diluting the reference fluid or gas which are configured to obtain reference fluids or gases comprising different respective concentrations of one or more contaminants from the reference gas in the reservoir 17.

According to another aspect of the present invention, the measurement station also comprises:
a first reservoir comprising a fluid having a predetermined concentration of one or more contaminants to be monitored, a first additional valve associated with a first mass flow meter for controlling the establishing of fluidic communication between the first reservoir and the analyser,
a second reservoir comprising a fluid that is free of the contaminant to be monitored, a second additional valve associated with a second mass flow meter for controlling the establishing of fluidic communication between the second reservoir and the analyser,
the control unit being configured to, during the verification of the accuracy of the analyser, control the opening of one of the first or second additional valve, to command a measurement of the concentration of the contaminant in the one of the first or second reservoir by the analyser, to control the closure of the one of the first or second additional valve, to control the opening of the other of the first or second additional valve, to command a measurement of the concentration of the contaminant in the other of the first or second reservoir by the analyser, to compare the measured values with the predetermined contaminant concentrations and to verify whether the difference between the measured values and the predetermined concentrations is lower than a predetermined maximum difference.

According to another aspect of the present invention, the control unit may be configured to command the measurement of the concentration in the first reservoir prior to the measurement of the concentration in the second reservoir. Other concentrations may also be obtained with the two reservoirs by diluting the contaminant of the first reservoir with the gas of the second reservoir; the mass flow meters associated with the first and the second reservoir can be used to carry out the desired dilution.

According to another aspect of the present invention, the control unit is configured to emit and/or send a warning signal in the event of a concentration of a contaminant greater than a predetermined threshold being detected by the analyser, the accuracy of which is verified. The warning signal is for example sent to a remote server such as a client server.

According to another aspect of the present invention, the measurement station may comprise a plurality of analysers disposed in parallel and a plurality of second controllable isolation valves interposed between the plurality of air inlets and the respective plurality of analysers so as to control the establishing of fluidic communication between the analysers and the air inlets.

According to another aspect of the present invention, the control unit is configured to control the opening and the closure of the first and second controllable isolation valves so as to establish fluidic communication between the sampling lines and the analysers successively and/or simultaneously according to a predetermined order.

According to another aspect of the present invention, the control unit is configured to command a verification of the accuracy of the analyser when a concentration of a contaminant greater than a predetermined threshold is detected by said analyser only for certain analysers.

According to another aspect of the present invention, the control unit is configured to emit and/or send a warning signal when a concentration of a contaminant greater than a predetermined threshold is detected and a verification of the accuracy is initiated. The warning signal is for example sent to a remote server such as a client server.

According to another aspect of the present invention, the air inlets comprise means for regulating the air flow rate such that the conveyance through the different sampling lines to which the air inlets are connected is the same regardless of their length and/or their diameter.

According to another aspect of the present invention, the means for regulating the air flow rate are valves with micro leaks, that is to say valves that allow the flow rate to be regulated in a precise manner.

According to another aspect of the present invention, during the verification of the accuracy of the analyser, if the difference between the measured concentration values and the predetermined concentrations is greater than a predetermined maximum difference, the control unit is configured to command a recalibration of the analyser.

The present invention also relates to a method for detecting airborne molecular contamination using a measurement station comprising a gas analyser and a plurality of air inlets configured to be in fluidic communication with the gas analyser and configured to be connected to one or more sampling lines, said method comprising the following steps:
establishing fluidic communication between sampling lines associated with the air inlets and the analyser, then measuring the concentration of one or more contaminants to be monitored in the sampling lines, the sampling lines being placed in fluidic communication with the analyser successively in order to measure the concentration of the contamination in the different sampling lines,
when the measurement of the concentration of a contaminant exceeds a predefined threshold, verifying the accuracy of the analyser.

According to another aspect of the present invention, when the accuracy of the analyser is verified, a warning signal is emitted by the measurement station or is sent to a remote server.

According to another aspect of the present invention, the measurement station comprises a plurality of analysers which allow a contaminant or a set of contaminants to be measured, and the analysers are placed in fluidic communication with the sampling lines simultaneously.

According to another aspect of the present invention, the step of verifying the accuracy of the analyser comprises establishing fluidic communication between the analyser and a first reservoir comprising a fluid having a predetermined concentration of a contaminant to be monitored and measuring the concentration by way of the analyser, then establishing fluidic communication between the analyser and a second reservoir comprising a fluid that is free of the contaminant to be monitored and measuring the concentration by way of the analyser.

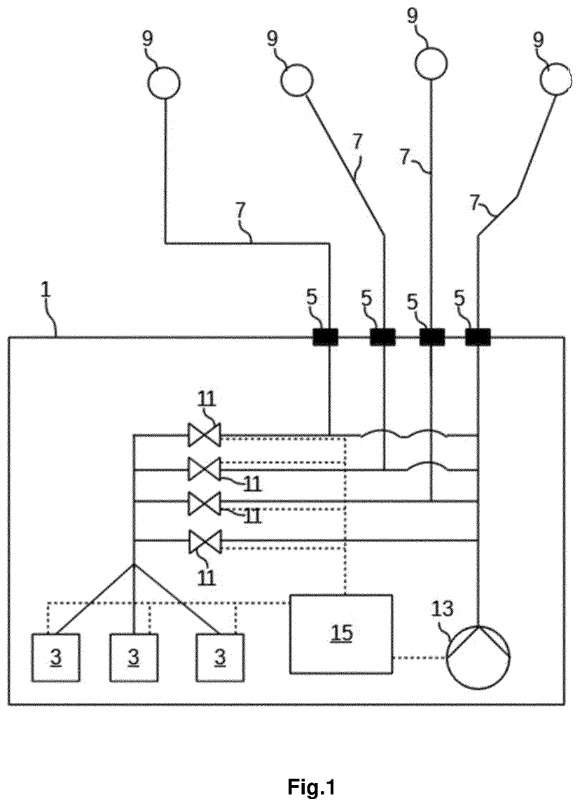

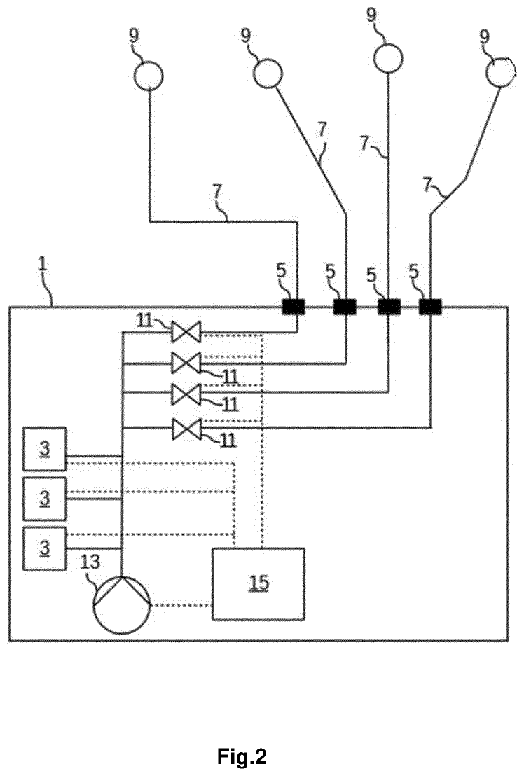

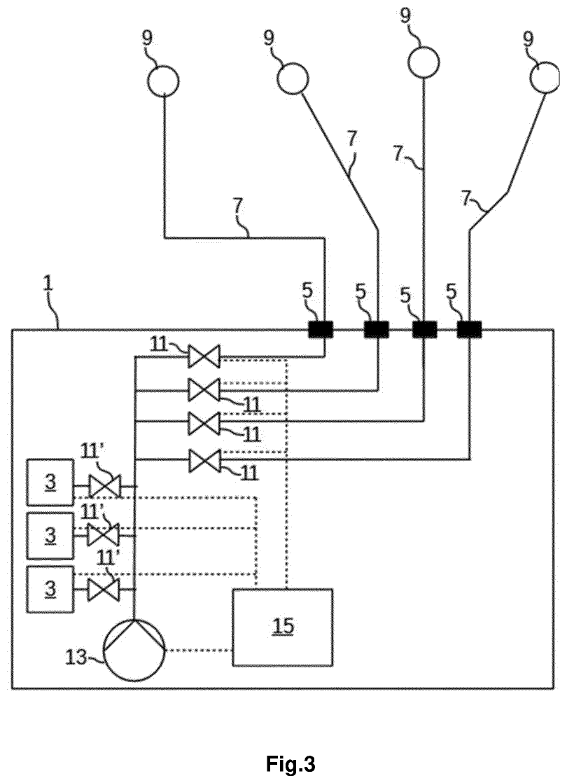

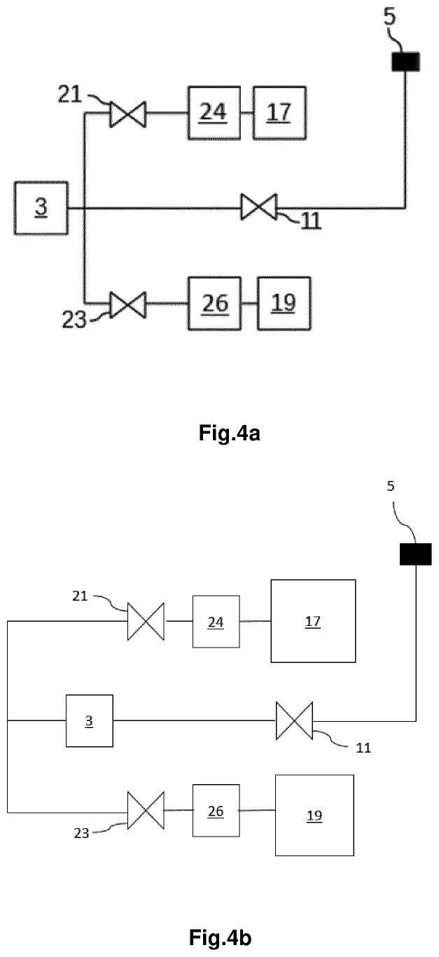

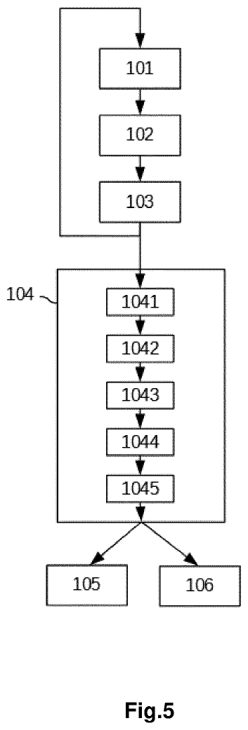

Other features and advantages of the invention will become more clearly apparent upon reading the following description, which is given by way of illustrative and non-limiting example, and from the appended drawings, in which:
FIG. 1 is a diagram of a measurement station and of the associated sampling lines according to a first embodiment;
FIG. 2 is a diagram of a measurement station and of the associated sampling lines according to a second embodiment;
FIG. 3 is a diagram of a measurement station and of the associated sampling lines according to a third embodiment;
FIG. 4a is a diagram of the reservoirs associated with an analyser according to a first embodiment;
FIG. 4b is a diagram of the reservoirs associated with an analyser according to a second embodiment;
FIG. 5 is a flow diagram of the steps of the method for detecting airborne molecular contamination using a measurement station.

In these figures, identical elements bear the same references.

The following embodiments are examples. Although the description refers to one or more embodiments, this does not necessarily mean that each reference relates to the same embodiment or that the features apply to a single embodiment. Individual features of different embodiments can also be combined or interchanged to provide other embodiments.

The present invention relates to a station for measuring airborne molecular contamination. FIG. 1 shows a diagram of such a measurement station 1. The measurement station 1 comprises a plurality of gas analysers 3, three in the present case. Each analyser 3 may be configured to measure the concentration of a predetermined contaminant or of a set of predetermined contaminants. The measurement station 1 also comprises a plurality of air inlets 5, four in the present case, which are configured to be placed in fluidic communication with the gas analysers 3. The fluidic communication between the air inlets 5 and the gas analysers 3 is for example realized by ducts. The air inlets 5 are configured to be connected to one or more sampling lines 7, notably one sampling line 7 per air inlet 5 as in the example of FIG. 1 (but one air inlet 5 may be connected to several sampling lines 7). That end of the sampling line which is on the opposite side from the air inlet 5 constitutes a sampling point 9. The sampling points 9 are for example distributed in different zones of a clean room.

In order to limit the influence of the length of the sampling lines 7 on the measurements by the analysers 3, the air inlets 5 may comprise means for regulating the air flow rate. These means for regulating the air flow rate are configured such that the conveyance through the different sampling lines 7 is the same regardless of their length. These means for regulating the air flow rate are for example valves with micro leaks which make it possible to ensure an identical flow rate for all of the sampling lines regardless of their length and/or their diameter.

The measurement station 1 also comprises a plurality of first controllable isolation valves 11 interposed respectively between the plurality of air inlets 5 and the gas analysers 3 so as to allow or prevent the establishing of fluidic communication between the air inlets 5 and the gas analysers 3. The first controllable isolation valves 11 may be realized by multiplexing valves which allow or prevent the establishing of fluidic communication between one or more air inlets 5 and one or more gas analysers 3. The measurement station 1 also comprises a conditioning pump 13 configured to suction air from the sampling lines 7 to the gas analysers 3. The conditioning pump 13 is for example connected to the air inlets 5 via ducts. As an alternative, the conditioning pump 13 may be placed downstream of the first controllable isolation valves 11 as shown in FIG. 2. In this embodiment, it may be necessary for at least one controllable isolation valve 11 to be open in order to ensure the proper operation of the conditioning pump 13. As a further alternative, a plurality of second controllable isolation valves 11′ may be disposed at the inlet of each analyser 3, as shown in FIG. 3, in order to select the analysers 3 to be connected to the air inlets 5. These second controllable isolation valves 11′ may also be used when the conditioning pump 13 is positioned as in the embodiment in FIG. 1. The analysers 3 may also comprise their own suction means, such as an internal sampling pump. In this case, it is not necessary to use the conditioning pump 13.

The measurement station 1 also comprises a control unit 15. The control unit 15 comprises, for example, a microcontroller or a microprocessor. The control unit 15 is configured to control the opening or the closure of the first isolation valves 11 and/or second isolation valves 11′ in order to establish or not establish fluidic communication between the gas analysers 3 and the sampling lines 7 so as to make it possible to analyse air from the different sampling points 9. The sampling lines 7 may be placed in fluidic communication one after the other according to a predetermined order. Certain sampling lines 7 may also be placed in fluidic communication with the gas analysers 3 simultaneously so as to simultaneously analyse air from several sampling points 9.

The control unit 15 is also connected to the conditioning pump 13 and to the analysers 3. The control unit 15 is also configured to command a verification of the accuracy of a gas analyser 3 when the concentration of a contaminant greater than a predetermined threshold is detected by this analyser 3 in order to avoid an incorrect detection of a contaminant.

To carry out the verification, as shown in FIG. 4a, the measurement station 1 also comprises a first reservoir 17 associated with the analyser 3, a first additional valve 21 and a first flow meter 24 (the first additional valve 21 and the first flow meter 24 may be combined in a single device corresponding to a mass flow rate controller) which are disposed between the first reservoir 17 and the analyser 3, a second reservoir 19 associated with the analyser 3, a second additional valve 23 and a second flow meter 26 (the second additional valve 23 and the second flow meter 26 may be combined in a single device corresponding to a mass flow rate controller) which are disposed between the second reservoir 19 and the analyser 3. The first reservoir 17 and second reservoir 19 are, for example, cylinders comprising pressurized gas. According to an alternative embodiment shown in FIG. 4b, the reservoirs 17, 19 are not fluidically connected to the pipe comprising the isolation valve 11 so as to avoid contamination of the sampling lines by the contaminant contained in the first reservoir 17.

The first reservoir 17 comprises a fluid having a first predetermined concentration of one or more contaminants to be monitored, for example benzene, the predetermined concentration of which may be between 1 and 100 ppb. The second reservoir 19 comprises a fluid having a second predetermined concentration of a contaminant to be monitored, in particular this second predetermined concentration may be zero such that the fluid in the second reservoir 19 is free of contaminant and contains, for example, clean air. The contents of the second reservoir 19 may then be used to dilute the contaminant contained in the first reservoir 17 and thus obtain a number of different predetermined concentrations greater than two with only two reservoirs. It is also possible to use a plurality of reservoirs containing different predetermined concentrations of the contaminant.

In practice, the first reservoir 17 and second reservoir 19 are associated with each analyser 3. A number of reservoirs 17, 19 greater than two may be associated with one analyser 3, the different reservoirs 17, 19 having different predetermined concentrations.

As an alternative, the measurement station 1 may also comprise a single reservoir 17 comprising a reference fluid having a predetermined concentration of one or more contaminants to be monitored and an additional valve 21 disposed between the reservoir 17 and the gas analyser 3, the control unit 15 being configured to, during the verification of the accuracy of the analyser 3, control the opening of the additional valve 21 and to command a measurement of the concentration of one or more contaminants in the reservoir 17. The measurement station 1 may also comprise means for diluting the reference fluid or gas which are configured to obtain reference fluids or gases comprising different respective concentrations of one or more contaminants from the reference gas in the reservoir 17.

The verification of the accuracy may be effected by injection of a clean fluid or gas (devoid of contaminant) or of a reference fluid or gas having a controlled (known) concentration of one or more contaminants (the reference gas may contain one or more compounds). The reference fluid or gas may also come from a permeation tube, which may or may not be heated.

Thus, when a concentration greater than a predetermined threshold is detected by an analyser 3, the control unit 15 commands that a verification of the accuracy of the analyser 3 be carried out. In practice, this automatic verification may be carried out for certain analysers 3. In addition, the concentration measurement may be repeated in order to verify that the predetermined threshold has been exceeded before a verification of the accuracy of the analyser 3 is initiated.

This verification comprises, as a first step, measuring, by way of the analyser 3, the concentration of the fluid contained in the first reservoir 17 via the opening of the first additional valve 21 and the closure of the other valves 11, 23 which are situated on ducts connected to the analyser 3, and, as a second step, measuring, by way of the analyser 3, the concentration of the fluid contained in the second reservoir 19 via the opening of the second additional valve 23 and the closure of the other valves 11, 21 which are situated on ducts connected to the analyser 3. The control unit 15 is also configured to compare the values measured by the analyser 3 with the predetermined concentrations. If the difference between the measured values and the predetermined concentrations is lower than a predefined maximum difference, the analyser 3 is considered to be reliable, that is to say that the accuracy is verified, and the detection of the contaminant is confirmed. The predefined maximum difference may be chosen by the user and input, for example via an interface linked to the control unit 15 such as a touchscreen or control buttons. The second reservoir may also be used to dilute the concentration of the first reservoir (the second reservoir is for example used to dilute the first when the second is clean air). The two additional valves 21 and 23 may then be open simultaneously in order to be able to deliver different concentrations. Different predetermined concentrations may then be transmitted successively to the analyser 3 to verify its accuracy.

The control unit 15 is then configured to emit a warning signal, such as a visual and/or audible signal, or send a warning signal, for example to a remote server.

In addition, a first warning signal may be emitted prior to the verification of the analyser 3 in order to indicate that the concentration of a contaminant greater than the predetermined threshold has been detected by the analyser 3 and a verification of the analyser 3 will be initiated.

By contrast, if during the verification of the accuracy of the analyser 3 the difference between the measured values and the predetermined concentrations is greater than the predefined maximum difference, the analyser 3 is considered to be unreliable such that the detection of the contaminant is not confirmed. The control unit 15 is then configured to initiate a recalibration of the analyser 3. As an alternative, a warning signal may be emitted by the control unit 15 in order to indicate the result of the verification and allow an operator to decide on the measures to be taken. Furthermore, the measurement, as a second step, of the fluid which is situated in the second reservoir 19 and which is free of contaminant makes it possible to not distort the subsequent measurements carried out by the analyser 3. In fact, the use of a gas that is free of contaminant makes it possible to purge the analyser 3, and even the ducts adjacent to the analyser 3.

The present invention also relates to a method for detecting airborne molecular contamination using a measurement station 1 as described above.

FIG. 5 shows a flow diagram of the steps of this method. The order of the steps or sub-steps may be different from the order presented and certain steps or sub-steps may be carried out simultaneously.

The first step 101 relates to the establishing of fluidic communication between one or more sampling lines 7 associated with the different air inlets 5 and one or more analysers 3. Fluidic communication is established by the control unit 15 by controlling the opening or the closure of different valves 11, 11′ of the measurement station 1.

The second step 102 relates to the measurement, by the analyser or analysers 3, of the concentration of one or more contaminants to be monitored in the sampling line or lines 7 in fluidic communication with the analyser or analysers 3. If several analysers 3 are used, each analyser 3 may be configured to measure the concentration of a different contaminant or set of contaminants than the other analysers 3.

The third step 103 relates to the comparison of that concentration value of a contaminant which is measured by the analyser 3 and a predefined threshold set by the user. Different thresholds associated with different actions or different warnings may be defined in order to allow better evaluation of the scale of the contamination.

Steps 101 to 103 are repeated according to a predefined order so as to place the different air inlets 5 (and therefore the different sampling lines 7) in fluidic communication with the analyser or analysers 3 successively, it being possible for several air inlets 5 to be placed in fluidic communication with the analyser or analysers 3 simultaneously. The contaminant concentration being measured and compared with the predefined threshold for each configuration of valves 11, 11′ before passing to the next configuration of valves 11, 11′.

When the measurement of the concentration of the contaminant exceeds the predefined threshold, the control unit 15 initiates the procedure for verifying the accuracy of the analyser 3, corresponding to step 104.

This step 104 comprises a first sub-step 1041 which relates to the closure of the valves 11, 11′ of the ducts connected to the analyser 3 whose accuracy is being verified, and the opening of the first additional valve 21 associated with this analyser 3. The second sub-step 1042 relates to the measurement of the concentration of the contaminant in the first reservoir 17 by the analyser 3. The third sub-step 1043 relates to the closure of the first additional valve 21 and the opening of the second additional valve 23. The fourth sub-step 1044 relates to the measurement, by the analyser 3, of the concentration of the contaminant in the second reservoir 19 or in a mixture between the first reservoir 17 and the second reservoir 19 in a predefined dilution proportion. The fifth sub-step 1045 relates to the comparison of the values measured by the analyser 3 with the expected contaminant concentrations in the first reservoir 17 and the second reservoir 19 or in a mixture of gases from the two reservoirs 17 and 19 (predefined dilution of the concentration of the first reservoir 17 with the second reservoir 19) and the comparison of these differences with the predetermined permitted maximum differences. If the differences are lower than the permitted maximum differences, the measurement by the analyser 3 can be considered to be reliable and that concentration value of the contaminant which is measured in step 102 and which is greater than the predetermined threshold is confirmed, and the method continues to step 105 in which a warning indicating this contaminant concentration can be emitted.

One or more new measurements of the sampling line 7 in which the concentration greater than the predetermined threshold has been measured may also be carried out to confirm the first measurement. These new measurements may also be carried out in a zone adjacent to the zone in which the contamination has been detected. If the contamination is detected in several analysed sampling lines 7 simultaneously, the new measurement may be carried out on one of these sampling lines 7 to improve the determination of the extent of the contamination zone.

As an alternative, it is also possible to start with the concentration measurement of the contaminant in the second reservoir 19 (or a mixture of the two reservoirs 17, 19), then measure the concentration in the first reservoir 17. Several reservoirs 17 comprising different contaminants or different concentrations of a contaminant could also be used.

If the differences between the measured values and the predefined concentrations are greater than the permitted maximum differences, the measurement by the analyser 3 can be considered to be unreliable, and the method continues to step 106 in which a recalibration of the analyser 3 can be requested and a warning indicating the unreliability of the analyser 3 can be emitted.

Carrying out a verification of the accuracy of the analyser 3 when a concentration of a contaminant greater than a predetermined threshold is measured by this analyser 3 makes it possible to reduce the number of instances of incorrect detection without it being necessary to verify the accuracy for each measurement by the analyser 3. Specifically, a measurement takes a few minutes, for example five minutes, whereas a verification of the accuracy may take a few tens of minutes, for example 20 minutes. The management of the contamination of the clean room is thus not as constraining (due to the reduction in the number of instances of incorrect detection) since the detection of contamination generally leads to the intervention of an operator to analyse the origin of the contamination and to remedy this contamination.

1-16. (canceled)

17. A station for measuring airborne molecular contamination, comprising:
at least one gas analyser configured to measure a concentration of at least one contaminant;
a plurality of air inlets configured to be in fluidic communication with the gas analyser and configured to be connected to one or more sampling lines;
a plurality of first controllable isolation valves interposed between the plurality of air inlets and the gas analyser; and
a control unit configured to control an opening and a closure of the first controllable isolation valves in order to place the gas analyser in fluidic communication with at least one sampling line,
wherein the control unit is also configured to command a verification of an accuracy of the analyser when the concentration of a contaminant greater than a predetermined threshold is detected by said analyser.

18. The measurement station according to claim 17, further comprising a reservoir comprising a reference fluid having a predetermined concentration of one or more contaminants to be monitored and an additional valve disposed between the reservoir and the gas analyser, the control unit being configured to, during the verification of the accuracy of the analyser, control the opening of the additional valve and to command a measurement of the concentration of one or more contaminants in the reservoir.

19. The measurement station according to claim 17, further comprising a first reservoir comprising a fluid having a predetermined concentration of one or more contaminants to be monitored, a first additional valve associated with a first mass flow meter to control establishing of fluidic communication between the first reservoir and the analyser, a second reservoir comprising a fluid that is free of contaminant to be monitored, a second additional valve associated with a second mass flow meter to control establishing of fluidic communication between the second reservoir and the analyser, the control unit being configured to, during the verification of the accuracy of the analyser, control the opening of one of the first or second additional valve, to command a measurement of the concentration of the contaminant in the one of the first or second reservoir by the analyser, to control the closure of the one of the first or second additional valve, to control the opening of the other of the first or second additional valve, to command a measurement of the concentration of the contaminant in the other of the first or second reservoir by the analyser, to compare the measured values with the predetermined contaminant concentrations and to verify whether a difference between the measured values and the predetermined concentrations is lower than a predetermined maximum difference.

20. The measurement station according to claim 19, wherein the control unit is configured to command the measurement of the concentration in the first reservoir prior to the measurement of the concentration in the second reservoir.

21. The measurement station according to claim 17, wherein the control unit is configured to emit a warning signal in an event of a concentration of a contaminant greater than a predetermined threshold being detected by the analyser, the accuracy of which is verified.

22. The measurement station according to claim 17, further comprising a plurality of analysers disposed in parallel and a plurality of second controllable isolation valves interposed between the plurality of air inlets and the respective plurality of analysers so as to control establishing of fluidic communication between the analysers and the air inlets.

23. The measurement station according to claim 22, wherein the control unit is configured to control the opening and the closure of the first and second controllable isolation valves so as to establish fluidic communication between the sampling lines and the analysers successively and/or simultaneously according to a predetermined order.

24. The measurement station according to claim 22, wherein the control unit is configured to command a verification of the accuracy of the analyser when a concentration of a contaminant greater than a predetermined threshold is detected by said analyser only for certain analysers.

25. The measurement station according to claim 17, wherein the control unit is configured to emit a warning signal when the concentration of the contaminant greater than the predetermined threshold is detected and the verification of the accuracy is initiated.

26. The measurement station according to claim 17, wherein the air inlets comprise means for regulating an air flow rate such that conveyance through the sampling lines to which the air inlets are connected is the same regardless of a length and/or a diameter of the sampling lines.

27. The measurement station according to claim 26, wherein the means for regulating the air flow rate are valves with micro leaks.

28. The measurement station according to claim 17, wherein, when a difference between the measured concentration and a predetermined concentration is greater than a predetermined maximum difference, the control unit is configured to command a recalibration of the analyser.

29. A method for detecting airborne molecular contamination using a measurement station comprising a gas analyser and a plurality of air inlets configured to be in fluidic communication with the gas analyser and configured to be connected to one or more sampling lines, said method comprising:
establishing fluidic communication, successively, between sampling lines associated with the air inlets and the analyser and measuring a concentration of one or more contaminants to be monitored in the sampling lines; and
verifying an accuracy of the gas analyser when the measurement of the concentration of the one or more contaminants exceeds a predefined threshold.

30. The detection method according to claim 29, wherein when the accuracy of the gas analyser is verified, a warning signal is emitted and/or sent to a remote server.

31. The detection method according to claim 29, wherein the measurement station comprises a plurality of gas analysers associated with a contaminant or a set of contaminants, and wherein the gas analysers are placed in fluidic communication with the sampling lines simultaneously.

32. The detection method according to claim 29, wherein the verifying the accuracy of the gas analyser comprises establishing fluidic communication between the gas analyser and a first reservoir comprising a fluid having a predetermined concentration of a contaminant to be monitored and measuring the concentration by way of the analyser, then establishing fluidic communication between the analyser and a second reservoir comprising a fluid that is free of the contaminant to be monitored and measuring the concentration by way of the analyser.
